(19) United States
(12) Patent Application Publication
(10) Pub. No.: US 2021/0308030 A1
(43) Pub. Date: Oct. 7, 2021

(54) SYSTEM, METHODS AND KITS FOR DIAGNOSIS AND TREATMENT OF FEMALE PATTERN HAIR LOSS

(71) Applicant: Follea International, Irvine, CA (US)

(72) Inventors: Ofer A. Goren, Irvine, CA (US); John McCoy, Irvine, CA (US)

(21) Appl. No.: 16/946,219

(22) Filed: Jun. 10, 2020

Related U.S. Application Data
(60) Provisional application No. 63/004,159, filed on Apr. 2, 2020.

(51) Int. Cl. A61K 8/63; A61Q 7/00

(52) U.S. Cl. CPC A61K 8/63; A61K 45/06; A61K 2800/805; A61Q 7/00

(57) ABSTRACT

Compositions and methods are disclosed herein for treating female pattern hair loss with phytoestrogens. A method for diagnosing subjects in need of a phytoestrogen treatment and guiding the application of a phytoestrogen treatment are also described. Additionally, a method for diagnosing a subject at high risk for female pattern hair loss who will likely respond to phytoestrogen prophylactic treatment is described. A method for combining the composition described herein with topical minoxidil for androgenetic alopecia is described.

### CROSS-REFERENCE

This application is a non-provisional utility application which claims priority to U.S. provisional application 63/004,159 filed Apr. 2, 2020, the entire contents of which are incorporated herein by reference.

### FIELD

The present invention relates to methods and compositions for the treatment of female pattern hair loss. Topical compositions containing soy isoflavones are described as well as a method of treatment. Additionally, a method for predicting female pattern hair loss and the likelihood a soy isoflavone treatment will be effective for treating hair loss are described.

### BACKGROUND

Female pattern hair loss (FPHL), also termed female androgenetic alopecia, is the progressive miniaturization of hair follicles on the female scalp. It is a frequently occurring condition affecting 30-40% of the female population by the age of 60. It is characterized by diffuse thinning of the crown region of the scalp while the frontal hairline remains intact. Addition signs of FPHL include wider thinning on the frontal scalp giving the balding area a triangular shaped figure resembling a “Christmas tree”. FPHL is the most common form of hair loss. The Sinclair scale for female pattern hair loss is commonly used to assess the degree of hair loss. Female pattern hair loss is hereditary but dependent on hormones to influence its development.

2% topical minoxidil is the only US FDA approved drug for the treatment of FPHL. It is estimated that 13-20% of FPHL patients experience a moderate increase in hair regrowth when using a 2% minoxidil mono-therapy. 5% minoxidil solutions are also used as a treatment for FPHL. Clinical trials suggest that 5% minoxidil has better efficacy than 2% minoxidil based on the average change in nonvellus hair count. Clinical studies in men suggest that approximately 40% of patients experience hair growth when using 5% minoxidil. The risk for adverse events with both 2% and 5% minoxidil is low; however, irritant dermatitis, allergic contact dermatitis, and hypertrichosis have been reported. Due to the prolonged treatment time required to elicit a therapeutic response (approximately 6 months) combined with the variable efficacy of minoxidil in the general population, an alternative treatment would have significant clinical utility.

Soy isoflavones are phytoestrogens found naturally in soybeans. Phytoestrogens are plant-derived compounds that have estrogen-like biologic activity. The most abundant isoflavones in soy beans are genistein and daidzein. Because of the ability to bind to the estrogen receptor, isoflavones have been studied as possible treatments for conditions mediated by the estrogen receptor, e.g., breast cancer and endometriosis.

The effects of isoflavones on estrogen mediated disease have had conflicting reports in literature. For example, isoflavones intake has been reported to have a protective effect against postmenopausal breast cancer in a Japanese population, while high serum isoflavone levels were associated with increased breast cancer rate in a United Kingdom population. It is clear that the mechanism of isoflavone interaction with estrogen mediated disease is complicated and likely dependent on genetic predisposition.

### SUMMARY

Compositions and methods are disclosed herein for treating female pattern hair loss (FPHL) with isoflavones. Because of the inter-individual variation in phytoestrogen sensitivity, the treatment method will require a diagnostic test to guide the selection of a phytoestrogen and a dosage. Additionally, a method is described to predict the risk that a subject will develop FPHL and the likelihood that an isoflavone will have a prophylactic effect to prevent FPHL.

In an exemplary embodiment, a method of treating alopecia in a subject in need thereof involves: applying to the scalp of the subject a composition containing a phytoestrogen.

In some embodiments, after applying the composition containing a phytoestrogen, the subject subsequently applies a topical minoxidil composition.

In some embodiments, the phytoestrogen is an isoflavone.

In some embodiments, the isoflavone is any one or combination of daidzein, genistein, glycitein, formononetin, biochanin A, daidzin, genistin, glycitin, ononin, sissotrin, acetyldaidzin, acetylgenistin, acetylglycitin, malonyldaidzin, malonylgenistin, malonylglycitin, malonylononin, or malonylsissotrin.

In an exemplary embodiment, a method to determine whether a subject will respond to or benefit from a phytoestrogen based treatment for alopecia involves: measuring a variant of SNP rs1013718 in the ESR2 gene of the subject.

In some embodiments, the method involves: collecting a DNA sample from the subject; extracting the DNA from the sample; amplifying DNA segments of the extracted DNA corresponding to a variant of SNP rs1013718 in the ESR2 gene; and analyzing the data to determine the SNP rs1013718 variant of the subject.

In some embodiments, the variant of SNP rs1013718 is any one or combination of “CC”, “CT” or “TT”.

In some embodiments, the method involves: determining that the subject will respond to or benefit from the phytoestrogen based treatment when the variant is “CC”.

In some embodiments, the method involves: determining that the subject has an increased risk of developing female pattern hair loss when the variant is “CC”.

In some embodiments, the method involves: determining that the subject has a lower risk of developing female pattern hair loss when the variant is “CT” or “TT”, as compared to the risk of developing female patter hair loss when the variant is “CC”.

In some embodiments, the DNA sample is a saliva sample.

In some embodiments, after extracting the DNA from the sample, the DNA is purified and subsequently quantified.

In some embodiments, the DNA is amplified by a real-time polymerase chain reaction protocol.

In some embodiments, the DNA is analyzed in an allelic discrimination plot.

In an exemplary embodiment, a composition for treating or preventing alopecia in a subject in need thereof includes: a phytoestrogen or a pharmaceutically acceptable salt or hydrate thereof.

In some embodiments, phytoestrogen is an isoflavone.

In some embodiments, the isoflavone is any one or combination of daidzein, genistein, glycitein, formononetin, biochanin A, daidzin, genistin, glycitin, ononin, sissotrin, acetyldaidzin, acetylgenistin, acetylglycitin, malonyldaidzin, malonylgenistin, malonylglycitin, malonylononin, or malonylsissotrin.

In some embodiments, the composition further comprises an exfoliating agent and/or a preservative.

In some embodiments, the composition is formulated as a topical composition.

In an exemplary embodiment, a kit for treating or preventing alopecia in a subject in need thereof includes: a topical composition including phytoestrogen or a pharmaceutically acceptable salt or hydrate thereof; and a topical composition including minoxidil.

### BRIEF DESCRIPTION OF THE FIGURES

FIG. 1 shows an exemplary process flow diagram that can be used for an embodiment of the method disclosed herein.

FIG. 2 shows an exemplary Allelic Discrimination Plot.

### DETAILED DESCRIPTION

Androgenetic alopecia (AGA) is a common dermatological condition affecting approximately 50% of the population by the age of 50. Currently, the only drug approved by the US Food and Drug Administration (FDA) for the treatment of AGA in both men and women is topical minoxidil. Clinical trials have demonstrated that after following 16 weeks of 5% minoxidil therapy, approximately 30-40% of patients regrow hair. As used herein, the terms “prevent” or “prevention” and other derivatives of the words, when used in reference to alopecia, e.g., androgenetic alopecia, refer to a reduced likelihood of alopecia in an individual receiving a given treatment relative to that of a similar individual at risk for alopecia but not receiving that treatment. As such, the terms “prevent” and “prevention” encompass a treatment that results in a lesser degree of alopecia, e.g., androgenetic alopecia, than would be otherwise expected for a given individual. Efficacy for prevention of alopecia, e.g., androgenetic alopecia, can be established through controlled studies, e.g., in which a subject is administered a treatment (e.g., a topical treatment) and another subject is administered a placebo. Under these circumstances, if the subject treated with the topical treatment undergoes less hair loss over time relative to the subject receiving the placebo, e.g., at least 5% less, at least 10% less, at least 15% less, at least 20% less, at least 25% less, at least 30% less, at least 35% less, at least 40% less, at least 45% less, at least 50% less or beyond, the treatment is effective for the prevention of alopecia, e.g., androgenetic alopecia.

As used herein, the term “subject” refers to a human in need of a therapeutic treatment for androgenetic alopecia or any other form of alopecia. In preferred embodiments, the subject is female.

As used herein, the terms “treat,” “treatment,” or “treating” refer to therapeutic treatments, wherein the object is to reverse, alleviate, ameliorate, inhibit, slow down or stop the progression or severity of a disease or condition, e.g., androgenetic alopecia or other form of alopecia. The term “treating” includes reducing or alleviating at least one adverse effect or symptom of a disease or condition, e.g., androgenetic alopecia or other form of alopecia. Treatment is generally “effective” if one or more symptoms are reduced. Alternatively, treatment is “effective” if the progression of a disease is reduced or halted. That is, “treatment” includes not just the improvement of symptoms, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the extent or amount of hair loss is reduced, or the progression of hair loss is slowed or halted. The term “treatment” of a disease also includes providing relief from the symptoms or side-effects of the disease (including palliative treatment).

As used herein the term “comprising” or “comprises” is used in reference to compositions, methods, etc. refers to component(s) or method steps that are present in the method or composition, yet allows for the composition, method, etc. to also include unspecified elements.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

As used herein the term “alopecia” refers to all forms of hair loss in men and women including but not limited to traction alopecia, androgenetic alopecia, male pattern baldness (MPB), female pattern hair loss (FPHL), alopecia areata, alopecia universalis, telogen effluvium, chemotherapy induced alopecia, hair shedding, eyebrow hair loss, beard hair loss and hair thinning. The term permits the presence of elements that do not materially affect the basic and novel or functional characteristic(s) of that embodiment

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Disclosed herein are methods to treat or prevent various forms of alopecia, e.g. female pattern hair loss or androgenetic alopecia. One aspect of these methods includes the use of a topical composition applied to the scalp that contains a phytoestrogen. In one embodiment, the method further includes the application of topical minoxidil composition subsequent to a topical composition applied to the scalp.

In one embodiment, a phytoestrogen is applied to a skin section, such as a section of the scalp which contains at least one hair follicle. In another embodiment, a phytoestrogen is applied to the scalp containing at least one hair follicle to treat androgenetic alopecia or FPHL. In one embodiment, the phytoestrogen is an isoflavone. Examples of isoflavones include, but are not limited to, daidzein, genistein, glycitein, formononetin, biochanin A, daidzin, genistin, glycitin, ononin, sissotrin, acetyldaidzin, acetylgenistin, acetylglycitin, malonyldaidzin, malonylgenistin, malonylglycitin, malonylononin, or malonylsissotrin.

The therapeutic agents, particularly the phytoestrogens, described herein and used in the present methods may be formulated into compositions according to the knowledge of one of skill in the art. In one embodiment, the phytoestrogens are formulated for topical slow or prolonged release.

The therapeutic agents, particularly the phytoestrogens, described herein and used in the present methods may be formulated into compositions according to the knowledge of one of skill in the art. In one embodiment, the phytoestrogen is encapsulated in order to increase the water solubility of the therapeutic agent. In another embodiment, the phytoestrogen is encapsulated in order to reduce the loss through degradation of therapeutic agent, for example, to reduce oxidation of the therapeutic agent.

In one embodiment genistein or daidzein is encapsulated to overcome its poor water solubility and facile oxidative degradation.

Another aspect of the present invention is a diagnostic test or method that is used to determine if a subject will likely respond to treatment with a phytoestrogen. In one embodiment a subject's likely response to a treatment containing a phytoestrogen is determined by measuring the variant of SNP rs1013718 in the ESR2 gene. In another embodiment, a subject can have one of three possible test results for variants of SNP rs1013718 in the ESR2 gene. In another embodiment, variants of SNP rs1013718 in the ESR2 gene can include “CC”, “CT” or “TT”. In one embodiment of the present invention a subject with a “CC” variant of SNP rs1013718 in the ESR2 gene is predicted to have a protective effect from a phytoestrogen treatment. In yet another embodiment of the present invention a subject with a “CC” variant of SNP rs1013718 in the ESR2 gene is predicted to have an increased risk for developing FPHL compared to women with the “CT” or “TT” variant.

One other aspect of the present invention is a diagnostic test or method that is used to determine if a subject will likely benefit from a prophylactic treatment with a phytoestrogen to prevent FPHL. In one embodiment, a subject's likely response to a prophylactic treatment containing a phytoestrogen is determined by measuring the variant of SNP rs1013718 in the ESR2 gene. In another embodiment, a subject likely to benefit from a prophylactic treatment with a phytoestrogen to prevent FPHL can have one of three possible test results for variants of SNP rs1013718 in the ESR2 gene. In another embodiment, variants of SNP rs1013718 in the ESR2 gene can include “CC”, “CT” or “TT”. In one embodiment, a subject with a “CC” variant of SNP rs1013718 in the ESR2 gene is predicted to benefit from a prophylactic treatment with a phytoestrogen to prevent FPHL. In yet another embodiment of the present invention a subject with a “CC” variant of SNP rs1013718 in the ESR2 gene is predicted to have an increased risk for developing FPHL compared to women with the “CT” or “TT” variant and will likely benefit from a prophylactic treatment with a phytoestrogen to prevent FPHL.

In another embodiment, the phytoestrogen is formulated in a shampoo, a foam, ointment, spray, solution, gel, slow release capsule, oral tablet, dry shampoo, or any similar compound or delivery vehicle or methodology. Topical application is preferred. In one embodiment, the composition is formulated in a topical cream. In another embodiment, the composition is formulated in a hair styling product selected from the group consisting of a styling gel, a styling foam, and a hair conditioner.

In another embodiment, the composition may comprise an exfoliating agent to promote abrasion of the surface of the scalp. Examples of the exfoliating agent include (1) inorganic and/or metallic particles such as: boron nitride, in body-centered cubic form (Borazon); aluminosilicate (e.g. nepheline); zircon; mixed oxides of aluminum such as emery; zinc oxide; aluminum oxides such as aluminas or corundum; titanium oxide; titanium oxide coated mica; carbides, in particular silicon carbide (carborundum); or other metal oxides; metals, and metal alloys such as iron shot, steel shot, and in particular perlite; silicates such as glass, quartz, sand, or vermiculite; calcium carbonate (e.g. Bora-Bora sand or Rose de Brignoles sand) or magnesium carbonate; sodium chloride; pumice stone; amorphous silica; diamond; ceramics, and (2) organic particles such as: fruit stones, in particular apricot stones, e.g. Scrubami® apricot; wood cellulose, e.g. ground bamboo stem; coconut shell, e.g. coconut exfoliator; polyamides, in particular Nylon-6; sugars; plastic microbeads, e.g. polyethylenes or polypropylenes; ground walnut; ground apricot seed; ground shells, and (3) mixed particles associating organic and inorganic compounds, and particles coated in the above compounds. The exfoliating agents may be in the form of microbeads of less than five millimeters in its largest dimension that have an exfoliating effect.

In another embodiment, the composition may comprise an exfoliating agent to promote absorption of the phytoestrogen into scalp. Example of the exfoliating agent include salicylic acid.

In one embodiment, the composition comprising a phytoestrogen can be formulated as a drug. In one embodiment, the composition comprising a phytoestrogen can be formulated as a cosmetic product

The amount of therapeutic agent present in the composition may be determined by one of skill in the art using known methodologies. In certain embodiments, the phytoestrogen is present in the composition in a concentration from about 0.0020% to 0.0030%, or about 0.0025% by weight. In another embodiment, the therapeutic agent such as a phytoestrogen is present in the composition in a concentration of about 0.0025%, 0.0033%, 0.005%, 0.01%, 0.02%, 0.025%, or 0.10% by weight.

In other embodiments, the therapeutic agent, such as the phytoestrogen, is present in the topical composition for use in the methods disclosed herein in a concentration from about 0.1% to 35%, about 1.0% to 30%, about 0.2% to 30%, about 0.2% to 25%, about 0.2% to 20%, about 0.2% to 15%, about 0.2% to 10%, about 0.2% to 5%, about 0.2% to 4%, about 0.2% to 3%, about 0.2% to 2%, about 0.2% to 1%, about 10.0% to 30%, about 15.0% to 30%, about 20.0% to 30%, about 10% to 20%, about 10% to 15%, about 15% to 20%, about 15% to 60%, about 20% to 60%, about 50% to 60%, and about 45% to 55% by weight.

In one embodiment, the composition comprises a phytoestrogen in a concentration of about 0.025%, about 0.033%, about 0.05%, about 0.1%, about 0.2%, about 0.25%, about 0.30%, about 0.40%, about 1.0%, about 1.5%, about 2.0%, or about 2.5% by weight.

The compositions used in the present disclosure, particularly compositions containing a phytoestrogen, may be formulated with a preservative such as EDTA (0.1-0.5% by weight of the formulation) and/or sodium metabisulfite (0.1-0.5% by weight of the formulation). In some embodiments, a penetration enhancer is selected from one or more of the group consisting of alcohols, glycols, fatty acids, fatty esters, fatty ethers, occlusive agents, surface active agents, dimethylaminopropionic acid derivatives, terpenes, sulfoxides, cyclic ethers, amides, and amines. Other components of the formulations used herein may be chosen from cosmetically approved excipients known in the art, including water, thickeners, etc.

The composition may be packaged in a kit with an applicator for application to the skin. An aspect of the invention is also directed to a kit comprising a composition of the therapeutic agent, such as a phytoestrogen, and an applicator, and to a kit comprising a composition of the therapeutic agent, such as a phytoestrogen, and a hair brush or comb, particularly a brush or comb that provides exfoliating effect on the scalp such that there is light abrasion after its use that enhances penetration of the therapeutic in the scalp. In one embodiment, the therapeutic agent is provided in a metered dose applicator that provides for a fixed volume of the composition to be administered with each administration, such as 1 ml of the topical composition per administration.

In another embodiment, the composition may be packaged in a kit including a topical minoxidil formulation. For example, a 2% minoxidil topical solution, a 3% topical minoxidil solution, a 5% topical minoxidil solution, a 5% topical minoxidil foam, a 10% topical minoxidil solution

It will be understood that the ranges described above, and throughout this document, are also intended to encompass single values contained within these ranges. For example, for a formulation comprising a particular ingredient in a range between 1-50%, a percentage of 5% or 49% is also intended to be disclosed.

### Therapeutic Agents

The methods of the present disclosure may be used with a phytoestrogen or other compound. Suitable phytoestrogens can be utilized including but are not limited to, daidzein, genistein, glycitein, formononetin, biochanin A, daidzin, genistin, glycitin, ononin, sissotrin, acetyldaidzin, acetylgenistin, acetylglycitin, malonyldaidzin, malonylgenistin, malonylglycitin, malonyl ononin, or malonylsissotrin. Additionally, derivatives of phytoestrogens can be utilized including derivatives of the compounds mentioned above. In other embodiments, a prodrug that is activated to become a phytoestrogen can be utilized.

In one embodiment, the phytoestrogen is genistein or daidzein, or a pharmaceutically acceptable salt or hydrate thereof, in a composition in a concentration of 0.0025% to 40%, 0.0025% to 25% by weight, or 0.005% to 22.5% by weight, or 0.0075% to 20% by weight, or 1% to 17.5% by weight, or 1.5% to 15% by weight, or 2% to 14.5% by weight, or 2.5% to 14% by weight, or 5% to 13.5% by weight, or 7.5% to 12.5% by weight, or 8% to 12% by weight, or 8.5% to 11.5% by weight, or 9% to 11% by weight, or 9.25% to 10.75% by weight, or 9.5% to 10.5% by weight, or 9.6% to 10.4% by weight, or 9.7% to 10.3% by weight, or 9.8% to 10.2% by weight, or 9.9% to 10.1% by weight, or 9.95% to 10.05% by weight, or 9.96% to 10.04% by weight, or 9.97% to 10.03% by weight, or 9.98% to 10.02% by weight, or 9.99% to 10.01% by weight.

In one embodiment, the phytoestrogen is genistein, or a pharmaceutically acceptable salt or hydrate thereof, in a composition in a concentration at a range of 0.25%, 0.5%, 0.75%, 1%, 1.5%, 2%, 2.5%, 5%, 7.5%, 8%, 8.5%, 9%, 9.25%, 9.5%, 9.6%, 9.7%, 9.8%, 9.9%, 9.95%, 9.96%, 9.97%, 9.98%, or 9.99% by weight as the lower weight limit of the range to an upper weight limit of 10.01%, 10.02%, 10.03%, 10.04%, 10.05%, 10.1%, 10.2%, 10.3%, 10.4%, 10.5%, 10.75%, 11%, 11.5%, 12%, 12.5%, 13.5%, 14%, 14.5%, 15%, 17.5%, 20%, 22.5%, 25%, 30%, 35%, 40%, 45%, or 50% by weight (e.g., a range of 0.25% to 10.01%, 0.25% to 10.02%, 0.5% to 10.01%, 0.5% to 10.02%, etc.).

In some embodiments, provided herein is a phytoestrogen formulated with a carrier or delivery vehicle optimized for delivery of the phytoestrogen to the scalp. A phytoestrogen can be released using several different formulations or release methods including time release, creams, ointments, sprays, capsules, or other release methods. For instance, the phytoestrogen can be incorporated into a shampoo for utilization during showering. In other embodiments, the phytoestrogen can be included in ointments or other topical creams that could be applied to the scalp so that it can be slowly absorbed into the skin. In other embodiments, the phytoestrogen can be included in a liquid spray or aerosol medium to be applied to the scalp. In other embodiments, the phytoestrogen can be incorporated into capsules or other slow release vehicles that would allow the chemical or agent to be slowly released into the dermis of the scalp. Capsules or vehicles that encapsulate the phytoestrogen can include, but are not limited to, liposomes, non-ionic liposomes, niosomes, novasome I, erythromycin-Zn complex, microspheres, nanoparticles, solid lipid nanoparticles, and nanoemulsions. In some embodiments, this can include a gel or foam that is applied to the scalp. It is specifically contemplated that the phytoestrogen can be formulated in hair care products such as a shampoo, styling gel, styling foam, hair conditioner, hair serum, a hair mask, etc.

Any of the aforementioned phytoestrogens can be used routinely, e.g., once daily, twice daily, every other day or once a week. Routine use of the phytoestrogen would be indicated as an adjuvant therapy for minoxidil in androgenetic alopecia patients. In a preferred embodiment, a composition (e.g., a shampoo) of any of the aforementioned phytoestrogens is used daily by a subject using minoxidil to increase the effectiveness of minoxidil.

Efficacy of treatment to treat or prevent androgenetic alopecia can be determined by monitoring the density of hairs on a given area of the subject's body, e.g., a given area of the scalp. If the rate of hair loss is reduced, e.g., by 10% or more following treatment, the treatment is effective for the prevention of androgenetic alopecia. Similarly, if hair density remains the same, the treatment is effective for the prevention of androgenetic alopecia. If the density of hair increases, e.g., by 5% or more, e.g., by 10% or more following treatment, the treatment is also considered effective for the treatment and/or prevention of androgenetic alopecia.

Efficacy of treatment to treat or prevent androgenetic alopecia can be determined by monitoring global photography. For example, the patient or an expert can assess the treatment response utilizing before and after global photographs.

As noted above, it is contemplated that all forms of alopecia can benefit from the technology described herein. For example, the technology described herein can be applicable to prevent or treat androgenic alopecia.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one or several features, while others specifically exclude one or several features, while still others mitigate a particular feature by inclusion of one or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that can have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

### EXAMPLES

### Example 1: Diagnostic Evaluation of Phytoestrogen Sensitivity

Indications for Use

The HairDx Test is indicated for reporting of the genetic variant SNP rs1013718 in the ESR2 gene. This report describes if a woman has phytoestrogen sensitivity and an increased risk of developing female pattern hair loss based on the genetic variant tested.

Clinical Performance

Approximately 85-88% of women with phytoestrogen sensitivity and female pattern hair loss (Sinclair grades 3, 4 or 5) have the “CC” variant of the SNP rs1013718.

However, many women with the “CC” variant of the SNP rs1013718 will not develop female pattern hair loss but will still benefit from treatment with phytoestrogens.

Analytical Performance

The accuracy of the HairDx Test was determined in comparison to bi-directional genetic sequencing. The results from the HairDx Test were greater than 99% correct.

Sample Test Results

DNA sample for a specific variant (SNP rs10137185) in the Oestrogen Receptor 2 (ESR2) Gene.

| Gene | Marker | Your Results* |
|---|---|---|
| ESR2 | SNP rs10137185 | CC |

Women with a similar genetic variation had an increased risk for developing female pattern hair loss and would benefit from treatment with phytoestrogens compared to women with the CT or TT variants.

| Gene | Marker | Your Results* |
|---|---|---|
| ESR2 | SNP rs10137185 | CT |

Women with a similar genetic variation did not have increased risk for developing female pattern hair loss and would not benefit from treatment with phytoestrogens.

| Gene | Marker | Your Results* |
|---|---|---|
| ESR2 | SNP rs10137185 | TT |

Women with a similar genetic variation did not have increased risk for developing female pattern hair loss and would not benefit from treatment with phytoestrogens.

Odds Ratios

Odds ratio describes the strength of the association between a genetic variant and the risk of developing female pattern hair loss and responding to phytoestrogen treatment. Women who carry the CC variant (odds ratio >1) have an increased risk for developing female pattern hair loss compared to women carrying the CT or TT variants (odds ratio=1).

| Genotype | Odds ratio |
|---|---|
| CC | 1.979 |
| CT or TT | 1.0 |

Likelihood Ratio

| | | 95% Confidence |
|---|---|---|
| Genotype | Likelihood ratio | interval |
| CC | 1.144 | 1.032 to 1.269 |
| CT or TT | 0.578 | 0.364 to 0.918 |

### Example 2: Procedure for Determining Phytoestrogen Sensitivity

Note: The following reference user manuals and instructions. Reference to these user manuals and instructions are to PureLink® Genomic DNA Kit User Manual (25-1012, Thermo fisher) and TaqMan® SNP Genotyping Assays User Guide (Publication Number MAN0009593 Revision B.0), each of which is incorporated herein by reference in their entirety.

Sampling Equipment

Saliva collection device—Oragene OCD100A/OGD 610 (DNA genotek Inc) in liquid transportation media.

TABLE 1
General Equipment

Equipment
Recommended nucleic acid extraction platform
Validated real-time thermal cycler
Class II hood/PCR workstation
Vortex mixer
Mini centrifuge
Water bath 50° C.
Centrifuge capable of processing 96 well plates
Fridge (2 to 8° C.) and freezer (−25° C. to −15° C.)

TABLE 2
Reagents

Reagents
Thermo Fisher Pure link DNA extraction kit
Positive extraction control (recommended e.g. previously characterized
positive sample)
Negative extraction control (recommended e.g. Nuclease free water)
Taqman probes and Master Mix

TABLE 3
Consumables

Consumables
Appropriate DNase/Rnase free plastic for DNA and PCR preparation
DNase/RNase free pipette tips
Disposable gloves, powderless
PCR plastic ware compatible with the thermo cycler of choice
Plate seals/ clear adhesive film

Real-Time PCR Instrumentation

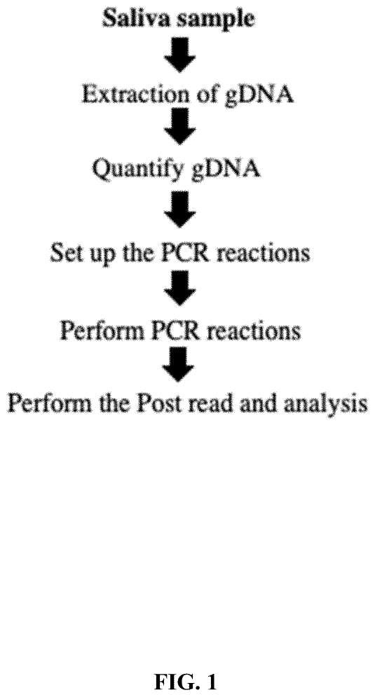

The HairDx test will be performed using the following real-time Polymerase chain reaction (PCR) instrument. See FIG. 1 for an exemplary process flow diagram.

TABLE 4
Real-Time PCR instrumentation specific details

| Real-Time PCR | Real-Time | |
|---|---|---|
| instrumentation | PCR platform | Validated consumables |
| manufacturer | Model | By Manufacturer |
| Applied | Quant Studio ™ 5 | 96-well, 96-well Fast, 384- |
| Biosystems | Real-Time PCR System | well, and 8-strip tubes |

The HairDx Test Workflow

Specimen Collection and DNA Extraction

The quality of the extracted DNA is inherently linked to the sensitivity by real-time PCR assay and therefore samples must be collected and stored following guidelines below:
Saliva samples are collected and handled according to the instructions provided with an ORA collect-Dx OGD-610 collection device.
DNA from collected saliva samples are extracted within 30 days after receiving.
DNA are extracted from the collection device using a PureLink™ Genomic DNA Mini Kit (Catalog #: K182002) extraction protocol.
Good laboratory practice recommends including at least one positive and negative extraction control per analysis.
Extracted samples are stored between −80° C. and −20° C. for long-term storage.

NOTE: Low concentrations of DNA can be unstable if stored for long periods.

TABLE 5
Recommended extraction protocols (commercial platforms)

| Extraction | Extraction | Extraction | Extraction elution |
|---|---|---|---|
| platform | kit | volume | volume |
| Purelink Genomic | Purelink | 500 μL transport | 50 μL |
| DNA | Genomic | media directly into | |
| | DNA Kit | the extraction | |
| | | platforms | |

TABLE 6
Required steps prior to extraction of DNA

| Purification Step | Notes |
|---|---|
| 1. Mix the sample in the DNA Genotek kit | This is to ensure that viscous samples are mixed |
| by inversion and gentle shaking for a few | properly |
| seconds. | |
| 2. Incubate the sample at 50° C. in a water | The heating step is crucial to make sure |
| bath for minimum of 1 hour and in air | DNA is released and that nucleases are |
| incubator for 2 hours. | permanently inactivated. |
| Note: The use of an air incubator maybe | This step may be achieved before |
| preferable to avoid floating of the tube in | purification. |
| water bath. | The entire sample must be incubated in |
| | original collection tube before aliquoting to |
| | ensure sample homogeneity. |
| | For convenience the sample may be |
| | incubated overnight at 50° C. |
| 3. Transfer 200 μL of the mixed sample to a | The remainder of the sample can be stored at |
| 1.5 mL microcentrifuge tube | room temperature or frozen (−15° C. to −20° C.) |
| 4. Proceed with extraction according to the | When the extraction is complete, stored the tubes |
| Purelink Mini DNA extraction protocol. | with DNA at 2-8° C., until DNA quantification is |
| | completed. |

DNA Quantification

1. Using the Biotek PowerWave XS, quantify the gDNA with the Take 3 microplate (UV/Vis spectrophotometry absorption measured at A260/A280 nm wavelength.
2. All steps for including installation, operation, calibration, cleaning and maintenance procedures are performed according to the manufacturer's instructions unless stated otherwise.
3. Vortex the gDNA sample tubes at MAX speed for 5 to 20 seconds. Using microcentrifuge, centrifuge the DNA sample tubes for 5 to 10 seconds to remove the liquid from the lids.
4. Blank the instrument using 2 μL of TE buffer.
5. Read 2 μL of each gDNA sample in duplicate.
6. Purified gDNA should have an A260/A280 ratio between 1.7 and 1.9 as determined with appropriate method.
7. If necessary, gDNA should be diluted in 10 mM Tris, pH 8.0 containing 0.1 mM EDTA (TE buffer-low EDTA) or nuclease-free H2O before use.

3.0 Test Procedure and PCR-Set Up

NOTE:
It is recommended to run each sample, positive or negative controls with each primer/probe master mix in adjacent wells e.g. Sample 1 is tested in reaction wells A1 and B1.
Before beginning PCR setup, samples are assigned to the appropriate wells within the appropriate plasticware; this information is recorded for traceability purposes.
Within the amplicon free area, thaw PCR reagents and TaqMan assay mix.
Briefly vortex and centrifuge the PCR Pro Amp master mix and each of the primer probes master mix.

3.1 Dilution of TaqMan Probes/Assays

Dilute the 40× or 80× predesigned and custom TaqMan® SNP Genotyping assays to a 20× working stock solution with 1×TE buffer.

NOTE: 1×TE buffer composition: 10 mM Tris HCl, 1 mM EDTA, pH 8.0, in DNase free, sterile filtered water.

NOTE: Store TaqMan reagents in the dark until ready to use. Add them last to reaction mixtures. Once added, keep the plate in the dark until the plate is disposed of following run on the thermocycler. Minimize freeze-thaw cycles.

3.2 Combine the Following Components for the Number of Reactions Required, Plus 10% Overage.
1. Dilute each DNA sample, including controls, in nuclease-free water to deliver 20 ηg per well. A final concentration of at least 0.2 ng/μL is required.
2. Add the following components according to Table 1
3. Volumes are given per reaction well and should be multiplied by the number of reaction wells, taking into account positive and negative controls. Also 2 additional reactions should be added in order to compensate for pipetting error. The prepared PCR reaction mastermixes should be thoroughly mixed and centrifuged for 10 seconds.

TABLE 7
Setup for PCR reactions per sample

| Assay Reagents | PCR mix (SNP) |
|---|---|
| PCR ProAMp mastermix with ROX | 12.5 μL |
| 20X Taqman assay working solution SNP1 | 1.25 μL |
| Total volume (per well) | 13.75 μL |

Example: For 10 samples genotyped for SNP, 1 positive control and 1 negative control, the final volume of the PCR Taqman master mix (SNP) will be 14× 14=193 μL (12 reactions and 2 extra to account for pipetting error):
For each sample or control, dispense 15 μL from the PCR mastermix for SNP being tested.
For each sample add 10 μL of extracted gDNA, sample to the well containing appropriate master mix for SNP.
For each positive control reaction, add 10 μL of positive control to the well containing SNP mastermix.
At least 1 positive control should be included with each mastermix to provide details on reaction efficiency.
For each negative control reaction, add 11.25 μL of negative control to a well contain SNP mastermix.

TABLE 8
Dispense volumes per reaction well of PCR

| Mastermix + Internal Control | PCR mastermix (SNP) | |
|---|---|---|
| PCR reaction mastermix Table 2 | 13.75 | μL |
| Sample/ Control material template | Upto 11.25 | μL * |
| Water | — | |
| Final Volume | 25 | μL |

* In case 20 ng of gDNA results in less than 11.25 μL, volume, add nuclease free water to bring up the total reaction volume to 25 μL

The plasticware should now be sealed with adhesive film then centrifuged briefly to bring the reaction mix to the bottom of the well and eliminate air bubbles. A non-optical seal can be used for this step. After centrifugation, transfer the plasticware to validated thermal cycler for amplication. Reference to the instrument manual should be made for instructions on setting up an amplification run. Amplification should be carried out according to instrument-specific parameters.

Thermal Cycler Conditions

Refer to the Quantstudio 5 Instruction manual for information on how to operate the Real-Time PCR instrument and perform data analysis and program the instrument following conditions described herein. It is important to visually inspect the amplification plots for each sample to ensure that the results recorded are due to true amplification and cannot be attributed to background noise recorded above the defined thresholds. Select the appropriate flourophore with each channel and assign to the relevant target.

Configure the Real Time PCR Instrument with the following settings:

Experiment type: Qualitative

Reagents used: TaqMan

Reagents Ramp Speed: Standard

Reaction volume: 25 μL

Passive Reference dye: ROX

TABLE 9
Thermal cycling conditions for the HairPGx test

| | Temperature | | | Number of | Data |
|---|---|---|---|---|---|
| Step | (° C.) | Time | | cycles | collection |
| Activation | 95 | 15 | min | 1 | off |
| Denaturation | 94 | 30 | s | 45 | off |
| Annealing/extension | 60 | 60 | s | | on |

TABLE 10
Detector channel used to detect the presence of the HairPGx
target SNPs

| | Green | Yellow | Orange | Red |
|---|---|---|---|---|
| Reporter Dye | FAM | JOE | ROX | Cy5 |
| Channel | | | | |
| Quencher | None | None | None | None |
| SNP 1 | Allele 1 | Allele 2 | | |

Interpretation of Results

Internal Control

Detection of an internal control is not required with a positive result. In instances where the internal control has failed but the sample has been reported as positive for one of the HairPDx SNPs the result should be considered valid. In cases where the sample is reported as negative for all targets and the internal control is negative, the assay should be repeated using the same sample but diluted 1:10. If the internal control is then positive previous result was due to a handling error/PCR inhibition and new retest results should be reported. In cases where the internal control is still reported as negative after retesting then sample should be re-tested starting from extraction step.

Analyze the Experimental Data

Follow the instructions for data analysis based on the instrument used.

TABLE 11
Instructions for data analysis

| Software | Features |
|---|---|
| Real-time instrument | Instrument software |
| software | View real-time trace data to aide |
| | genotype calling |
| | Data analysis varies depending on |
| | the real-time PCR system. |
| | See the instrument user guide for |
| | more information |
| TaqMan Genotyper | Desktop software |
| software | Create studies |
| | Overlay data from multiple plates |
| | View real-time trace data to aide |
| | genotype calling |

(TaqMan ® SNP Genotyping Assays User Guide (Publication Number MAN0009593 Revision B.0)

Instructions for Using Quantstudio Desktop and Analysis Software for Making Automatic Calls.

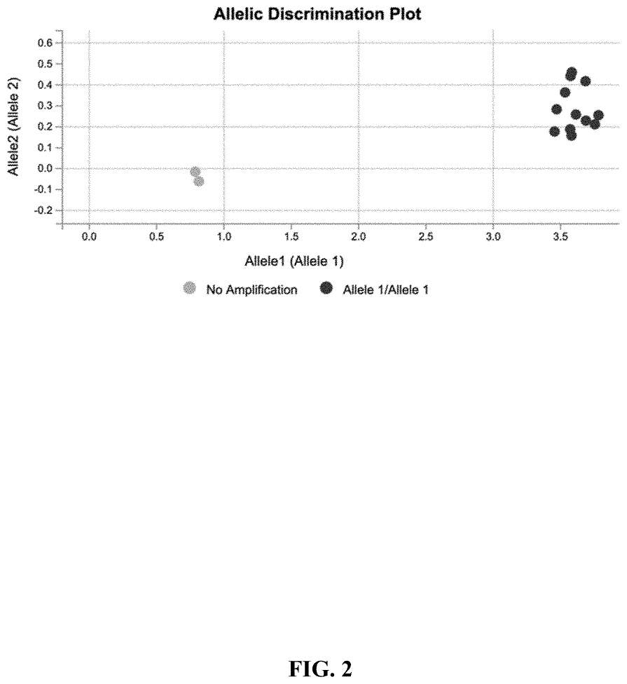

Allelic discrimination plot (see FIG. 2) can be viewed under the Results tab. In case there is no data displayed in the Results tab, click Analyze.
1. Under the Results tab using the dropdown option, select Allelic Discrimination Plot.
2. Plot can be configured by clicking on configure plot and making selections as follows: SNP Assay: Assay of interest can be selected, Plot Type: Select either Cartesian or Polar. Once this is done, the allelic discrimination plot is displayed for the selected SNP assay.
NOTE: All points in the plot are cyan because all the wells are selected in Plate Layout, by clicking anywhere in the plot or on Plate Layout the wells can be deselected. By doing this the data points in the plot change to the call colors.
3. Confirming the control data clusters as expected.
   a. Select the wells containing control under the Well Table or Plate Layout to highlight the respective data points in the plot.
   b. Check that the data points for each genotype control cluster along the expected axis of the plot.
4. In order to confirm only negative control wells are selected click on the cluster at the bottom left corner of the plot and make sure only corresponding wells are selected in the Plate Layout or Well Table.
It is possible that samples can unexpectedly cluster with the negative controls due to following reasons: samples contain no DNA, samples contain PCR inhibitors and/or samples are homozygous for a sequence deletion.
5. In order to review other clusters in the plot, follow the following steps:
a. Create a box around a cluster by Click-drag a box to select associated wells.
b. Confirm by checking if corresponding wells are selected in the Plate Layout or Well Table.
6. Outliers can be detected if the sample result falls outside the three genotype clusters. In case of outliers detected the results should be confirmed by performing a re-test for outliers as well as the samples that failed to amplify.

Instructions for Making Manual Calls:

Manual calls can be performed under the Results tab.
1. Select, Allelic Discrimination Plot using the dropdown menu.
2. In case the screen doesn't show data analyzed, click on the Analyze option.
3. Under the Allelic Discrimination Plot, using the lasso tool samples can be selected for making manual calls.
5. Click [symbol] , button and select the allele call using the apply call dropdown option.
6. Click Analyze.

TABLE 12
Cluster Assignment in an allelic discrimination plot

| Content of Samples | Location in AD plot |
|---|---|
| Allele 1 (homozygous, | Bottom right comer |
| labeled with VIC dye) | |
| Allele 2 (homozygous, | Top right corner |
| labeled with FAM dye) | |
| Alleles 1 and 2 | Approximately midway |
| (Heterozygous) | |
| No template control | Lower left comer |
| Undetermined | Anywhere outside the |
| | regions described above |
| No amplification | With NTC cluster in the |
| | lower left comer |

(TaqMan ® SNP Genotyping Assays User Guide (Publication Number MAN0009593 Revision B.0)

1. A method of treating alopecia in a subject in need thereof, the method comprising:
applying to the scalp of the subject a composition containing a phytoestrogen.

2. The method of claim 1, wherein after applying the composition containing a phytoestrogen, the subject subsequently applies a topical minoxidil composition.

3. The method of claim 1, wherein the phytoestrogen is an isoflavone.

4. The method of claim 3, wherein the isoflavone is any one or combination of daidzein, genistein, glycitein, formononetin, biochanin A, daidzin, genistin, glycitin, ononin, sissotrin, acetyldaidzin, acetylgenistin, acetylglycitin, malonyldaidzin, malonylgenistin, malonylglycitin, malonylononin, or malonylsissotrin.

5. A method to determine whether a subject will respond to or benefit from a phytoestrogen based treatment for alopecia, the method comprising:
measuring a variant of SNP rs1013718 in the ESR2 gene of the subject.

6. The method of claim 5, further comprising:
collecting a DNA sample from the subject;
extracting the DNA from the sample;
amplifying DNA segments of the extracted DNA corresponding to a variant of SNP rs1013718 in the ESR2 gene; and
analyzing the data to determine the SNP rs1013718 variant of the subject.

7. The method of claim 5, wherein the variant of SNP rs1013718 is any one or combination of “CC”, “CT” or “TT”.

8. The method of claim 7, further comprising:
determining that the subject will respond to or benefit from the phytoestrogen based treatment when the variant is “CC”.

9. The method of claim 7, further comprising:
determining that the subject has an increased risk of developing female pattern hair loss when the variant is “CC”.

10. The method of claim 9, further comprising:
determining that the subject has a lower risk of developing female pattern hair loss when the variant is “CT” or “TT”, as compared to the risk of developing female pattern hair loss when the variant is “CC”.

11. The method of claim 6, wherein the DNA sample is a saliva sample.

12. The method of claim 6, wherein after extracting the DNA from the sample, the DNA is purified and subsequently quantified.

13. The method of claim 6, wherein the DNA is amplified by a real-time polymerase chain reaction protocol.

14. The method of claim 6, wherein the DNA is analyzed in an allelic discrimination plot.

15. A composition for treating or preventing alopecia in a subject in need thereof, comprising:
a phytoestrogen or a pharmaceutically acceptable salt or hydrate thereof.

16. The composition of claim 15, wherein phytoestrogen is an isoflavone.

17. The composition of claim 16, wherein the isoflavone is any one or combination of daidzein, genistein, glycitein, formononetin, biochanin A, daidzin, genistin, glycitin, ononin, sissotrin, acetyldaidzin, acetylgenistin, acetylglycitin, malonyldaidzin, malonylgenistin, malonylglycitin, malonylononin, or malonylsissotrin

18. The composition of claim 15, wherein the composition further comprises an exfoliating agent and/or a preservative.

19. The composition of claim 15, wherein the composition is formulated as a topical composition.
